(19) United States
(12) Patent Application Publication
(10) Pub. No.: US 2022/0175729 A1
(43) Pub. Date: Jun. 9, 2022

(54) PHARMACEUTICAL USE OF CHLOROPHYLLIDE FOR CANCER THERAPY

(71) Applicant: I-SHOU UNIVERSITY, Kaohsiung City (TW)

(72) Inventors: Chih-Hui YANG, Kaohsiung City (TW); Jei-Fu SHAW, Kaohsiung City (TW); Yi-Ting WANG, Kaohsiung City (TW); Ting-Yu HUANG, Kaohsiung City (TW); Mi-Hsueh TAI, Kaohsiung City (TW); Pei-Han SHIH, Kaohsiung City (TW); Ru-Han SIE, Kaohsiung City (TW)

(21) Appl. No.: 17/537,545

(22) Filed: Nov. 30, 2021

(30) Foreign Application Priority Data
Dec. 7, 2020 (TW) 109143029

(51) Int. Cl. A61K 31/409; A61K 36/61; A61K 36/77; A61K 36/39; A61K 31/136; A61P 35/00; A61K 36/185; A61K 36/58; A61K 36/22; A61K 36/73; A61K 31/704; A61K 36/906

(52) U.S. Cl. CPC A61K 31/409; A61K 36/61; A61K 36/77; A61K 36/39; A61K 31/136; A61P 35/00; A61K 2236/333; A61K 36/58; A61K 36/22; A61K 36/73; A61K 31/704; A61K 36/906; A61K 2236/39; A61K 36/185

(57) ABSTRACT

The present invention provides a method for treating cancer, the method including the step of: administering a therapeutically effective concentration of chlorophyllide to a subject in need thereof. The present invention further provides a method for treating cancer, the method including the step of: administering a composition to a subject in need thereof, wherein the composition includes: a therapeutically effective concentration of chlorophyllide and a therapeutically effective concentration of anthracycline.

### CROSS REFERENCE

This non-provisional application claims priority of Taiwan Invention Patent Application No. 109143029, filed on Dec. 7, 2020, the contents thereof are incorporated by reference herein.

### FIELD OF THE INVENTION

The present invention is directed to pharmaceutical use of a chlorophyll derivative for cancer therapy, and more particularly to pharmaceutical use of chlorophyllide for cancer therapy.

### BACKGROUND OF THE INVENTION

Plants are the foundation of traditional medicines. A number of plant extracts possess anti-cancer properties, including Annona muricata L., Carica papaya, Colocasia gigantea, Annona squamosa Linn, Murraya koenigii L., Olea europaea L., Pandanus amaryllifolius Roxb., Chenopodium quinoa, Toona sinensis, Myristica fragrans, Thermopsis rhombifolia, and Cannabis sativa. The potential anti-cancer activities of these plants are associated with various bioactive compounds, including chlorophyll, pheophorbide, alkaloid, terpenoid, polysaccharide, lactone, flavonoid, carotenoid, glycoside, and cannabidiol. Beside the possibility of anti-cancer functions, compounds in plant extract demonstrate to exert function of anti-oxidation, anti-inflammation and attenuate side effects induced by chemotherapeutics. Additionally, those bioactive factors, especially chlorophyll and its derivatives, demonstrate potential for the treatment of cancer.

Chlorophyll, the most abundant pigment on earth, is present at high levels in green leafy plants, algae, and cyanobacteria. The catabolic derivatives of chlorophyll are chlorophyllide (chlide), pheophytin, pheophorbide, and phytol. Studies demonstrate that chlorophyll can reduce the growth and proliferation of MCF-7 breast carcinoma cells. Chlorophyll is also reported to promote cell differentiation, and to induce cell cycle arrest and apoptosis in HCT116 colon cancer cells. Chlorophyllide a/b and pheophorbide a/b are reported to reduce hydrogen peroxide-induced strand breaks and oxidative damage, and aflatoxin B 1-DNA adduct formation in hepatoma cells. Chlorophyllide is shown to decrease the levels of hepatitis B virus without affecting cell viability and viral gene products in tetracycline-inducible HBV-expressing HepDE19 cells. In human lymphoid leukemia molt 4B cells, pheophorbide a and phytol are able to induce programmed cell death. Phytol can also reduce inflammation by inhibiting neutrophil migration, reducing the levels of interleukin-1β (IL-1β), tumor necrosis factor-α (TNF-α), and oxidative stress. Pheophorbide a, in photodynamic therapy, is found to increase the levels of cytosolic cytochrome c, and is also tested against human pancreatic cancer cells (Panc-1, Capan-1, and HA-hpc2), hepatocellular carcinoma cells (Hep 3B), uterine sarcoma cells, human uterine carcinoma cells, and Jurkat leukemia cells. Pheophorbide is also shown to decrease the levels of procaspase-3 and -9 in Hep3B, Hep G2, and human uterine sarcoma MES-SA cells.

Extensive studies are performed with chlorophyllin (chllin, Cu-chl). Chlorophyllin, a semisynthetic, Cu-coupled, and water-soluble derivative of chlorophyll, is shown to significantly decrease the growth of mutagen-induced cancer cells. In vitro and in vivo studies are suggested that chlorophyllin possesses anti-genotoxic functions against compounds present in cooked meat, including N-nitroso compound and fungal toxin, aflatoxin B1 (AFB1), and dibenzo[d,e,f,p]chrysene (DBC). The regulation of cancer growth by chlorophyllin seems to involve the deactivation of key signal transduction pathways, including the nuclear factor kappa B, Wnt/b-catenin, phosphatidylinositol-3-kinase/Akt, and expressed E-cadherin and alkaline phosphatase pathways.

The amount of chlorophyll degraded globally each year is estimated to exceed 1000 million tons, and this is mostly derived from agriculture and food processing waste. Except for the edible parts of vegetables and fruits, most chlorophyll from low-value agricultural waste can only be degraded naturally. By using low-value agricultural waste as sources to collect chlorophyll, the cost of extraction can be reduced and maximum value of agriculture waste can be reached. Therefore, agricultural waste is potentially useful in the biomedical industry as a high-value nutraceutical and pharmaceutical material.

### SUMMARY OF THE INVENTION

The present invention is made based on the discovery that a product obtained by treating a plant leaf extract with chlorophyllase exhibits the activity of inhibiting the cancer cell survival, wherein its active ingredient at least includes chlorophyllide.

The present invention is made based on the discovery that the combination of doxorubicin and a product obtained by treating a plant leaf extract with chlorophyllase create a synergistic effect on the activity of inhibiting the cancer cell survival, wherein its active ingredient at least includes chlorophyllide.

Therefore, an embodiment of the present invention provides a method for treating cancer, the method including the step of: administering a therapeutically effective dose of chlorophyllide to a subject in need thereof.

Preferably, the cancer is breast cancer, liver cancer, colon adenocarcinoma, glioblastoma, lung cancer, buccal cancer, stomach cancer, colorectal cancer, nasopharyngeal cancer, skin cancer, kidney cancer, brain cancer, prostate cancer, ovarian cancer, cervical cancer, intestinal cancer, or bladder cancer.

Preferably, the cancer is drug-resistant cancer.

Preferably, the cancer is anthracycline-resistant cancer.

Preferably, the anthracycline-resistant cancer is doxorubicin-resistant cancer, daunorubicin-resistant cancer, arubicin-resistant cancer, epirubicin-resistant cancer, idarubicin-resistant cancer, valrubicin-resistant cancer, or mitoxantrone-resistant cancer.

Preferably, the cancer is triple-negative breast cancer.

Another embodiment of the present invention provides a method for treating cancer, the method including the step of: administering a product obtained by treating a plant leaf extract with chlorophyllase to a subject in need thereof, wherein the product comprises a therapeutically effective dose of chlorophyllide.

Preferably, the product is produced by the following steps of: providing plant leaves; performing extraction on the plant leaves with a solvent to obtain a crude extract; and treating the crude extract with chlorophyllase to obtain the product.

Preferably, the solvent is ethanol or hexane.

Preferably, the cancer is breast cancer, liver cancer, colon adenocarcinoma, glioblastoma, lung cancer, buccal cancer, stomach cancer, colorectal cancer, nasopharyngeal cancer, skin cancer, kidney cancer, brain cancer, prostate cancer, ovarian cancer, cervical cancer, intestinal cancer, or bladder cancer.

Preferably, the cancer is drug-resistant cancer.

Preferably, the cancer is anthracycline-resistant cancer.

Preferably, the anthracycline-resistant cancer is doxorubicin-resistant cancer, daunorubicin-resistant cancer, arubicin-resistant cancer, epirubicin-resistant cancer, idarubicin-resistant cancer, valrubicin-resistant cancer, or mitoxantrone-resistant cancer.

Preferably, the cancer is triple-negative breast cancer.

Another embodiment of the present invention provides a pharmaceutical composition, the composition including: a therapeutically effective dose of chlorophyllide and a therapeutically effective dose of anthracycline.

Preferably, the anthracycline is doxorubicin, daunorubicin, arubicin, epirubicin, idarubicin, valrubicin, or mitoxantrone.

Yet another embodiment of the present invention provides a pharmaceutical composition, the composition including: a product obtained by treating a plant leaf extract with chlorophyllase and a therapeutically effective dose of anthracycline, wherein the product comprises a therapeutically effective dose of chlorophyllide.

Preferably, the anthracycline is doxorubicin, daunorubicin, arubicin, epirubicin, idarubicin, valrubicin, or mitoxantrone.

Preferably, the product is produced by the following steps of: providing plant leaves; performing extraction on the plant leaves with a solvent to obtain a crude extract; and treating the crude extract with chlorophyllase to obtain the product.

Preferably, the solvent is ethanol or hexane.

Yet another embodiment of the present invention provides a method for treating cancer, the method including the step of: administering any of the foregoing compositions to a subject in need thereof.

Preferably, the therapeutically effective dose of chlorophyllide is from 12.5 to 100 μg/mL, and the therapeutically effective dose of anthracycline is from 0.625 to 20 μg/mL.

Preferably, the cancer is breast cancer, liver cancer, colon adenocarcinoma, glioblastoma, lung cancer, buccal cancer, stomach cancer, colorectal cancer, nasopharyngeal cancer, skin cancer, kidney cancer, brain cancer, prostate cancer, ovarian cancer, cervical cancer, intestinal cancer, or bladder cancer.

Preferably, the cancer is drug-resistant cancer.

Preferably, the cancer is anthracycline-resistant cancer.

Preferably, the anthracycline-resistant cancer is doxorubicin-resistant cancer, daunorubicin-resistant cancer, arubicin-resistant cancer, epirubicin-resistant cancer, idarubicin-resistant cancer, valrubicin-resistant cancer, or mitoxantrone-resistant cancer.

Preferably, the cancer is triple-negative breast cancer.

### BRIEF DESCRIPTION OF THE DRAWINGS

FIGS. 1A to 1C show the chlorophyll and chlorophyllide in products obtained by treating ethanol crude extracts from various plant leaves with chlorophyllase by using HPLC;

FIGS. 2A to 2C show the cytotoxicity effects on various cells of chlorophyllase-treated ethanol crude extracts from various plants by using MTT assay;

FIG. 3 shows the correlation between the contents of chlorophyllase-treated ethanol crude extracts from various plants and the cytotoxicity effects on various cells by using the Pearson correlation coefficient;

FIG. 4 shows the IC₅₀ values of chlorophyllase-treated ethanol crude extract from sweet potato, non-treated ethanol crude extract from sweet potato, and chlorophyllin for the cytotoxicity against various cells;

FIG. 5 shows the free radical scavenging rates of chlorophyllase-treated ethanol crude extract from sweet potato, non-treated ethanol crude extract from sweet potato, and chlorophyllin by using DPPH assay;

FIG. 6A shows the cellular cytotoxicity effects of various concentrations of doxorubicin by using MTT assay;

FIG. 6B shows the cellular cytotoxicity effects of various chlorophyllide concentrations in the chlorophyllase-treated hexane crude extract from sweet potato by using MTT assay;

FIG. 6C shows the cellular cytotoxicity effects of various chlorophyllide concentrations in the chlorophyllase-treated hexane crude extract from sweet potato in combination of the treatment of 0.625 μg/mL doxorubicin by using MTT assay;

FIG. 6D shows the cellular cytotoxicity effects of various doxorubicin concentrations in combination of the treatment of 100 μg/mL chlorophyllide in the chlorophyllase-treated hexane crude extract from sweet potato by using MTT assay;

FIG. 7 shows the number of differentially expressed genes identified from MCF7 breast cancer cells treated with chlorophyllide relative to reference untreated MCF7 breast cancer cells and that identified from MDA-MB-231 breast cancer cells treated with chlorophyllide relative to reference untreated MDA-MB-231 breast cancer cells by using NGS;

FIG. 8A shows the classification of differentially expressed genes in MCF7 breast cancer cells treated with chlorophyllide relative to reference untreated MCF7 breast cancer cells by using the Gene Ontology analysis;

FIG. 8B shows the classification of differentially expressed genes in MDA-MB-231 breast cancer cells treated with chlorophyllide relative to reference untreated MDA-MB-231 breast cancer cells by using the Gene Ontology analysis;

FIG. 9A shows the classification of differentially expressed genes in MCF7 breast cancer cells treated with chlorophyllide relative to reference untreated MCF7 breast cancer cells by using the KEGG pathway enrichment analysis;

FIG. 9B shows the classification of differentially expressed genes in MDA-MB-231 breast cancer cells treated with chlorophyllide relative to reference untreated MDA-MB-231 breast cancer cells by using the KEGG pathway enrichment analysis;

FIG. 10 shows the expression levels of differentially expressed genes identified from MCF7 breast cancer cells treated with chlorophyllide relative to reference untreated MCF7 breast cancer cells and those identified from MDA-MB-231 breast cancer cells treated with chlorophyllide relative to reference untreated MDA-MB-231 breast cancer cells by using NGS;

FIG. 11A is a bar graph illustrating the expression levels of exemplary differentially expressed genes identified from MCF7 breast cancer cells treated with chlorophyllide relative to reference untreated MCF7 breast cancer cells by using NGS and RT-PCR; and

FIG. 11B is a bar graph illustrating the expression levels of exemplary differentially expressed genes identified from MDA-MB-231 breast cancer cells treated with chlorophyllide relative to reference untreated MDA-MB-231 breast cancer cells by using NGS and RT-PCR.

### DETAILED DESCRIPTION OF THE INVENTION

The detailed description and preferred embodiments of the invention will be set forth in the following content, and provided for people skilled in the art to understand the characteristics of the invention.

The compound chlorophyllide used in the content is represented by a general formula (I)

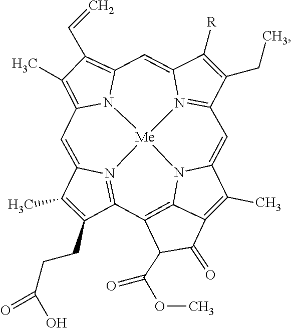

in which Me is a Mg atom, and R is a CH₃ group or a CHO group. Generally, while R is a CH₃ group, the compound is called “chlorophyllide a”; while R is a CHO group, the compound is called “chlorophyllide b”.

### Example 1: Preparation of Crude Extracts and Chlorophyll Extraction

The leaves of guava, sweet potato, banana, Chinese toona, logan, wax apple, mango, caimito, and cocoa were used to extract chlorophyll. 10 g (wet weight) of leaves were washed, dried, and ground into powder with a pestle and mortar. Leaf mixtures were then frozen in liquid nitrogen and stored at −80° C. in a deep freezer. Chlorophyll was extracted by immersing leaves in ethanol solvent (or hexane solvent) for 48 h. Ethanol crude extracts (or hexane crude extracts) from leaves were centrifuged at 1500 g for 5 min and keep at −20° C. for further experiments. To measure the concentrations of chlorophyll a/b, the crude extracts were passed through a 0.22-μm filter and the absorbance was measured at 649 and 665 nm, which were the major absorption peaks of chlorophyll a and b, respectively. The estimated concentrations of chlorophyll a and b in crude extracts were calculated according to the following equation:

chlorophyll a concentration (n/mL)=13.7×A665−5.76×A649;

chlorophyll b concentration (n/mL)=25.8×A665−7.6×A649.

The chlorophyll a/b concentrations in crude extracts calculated with the empirical equation were multiplied by the volume of the solvent that resulted in the relative chlorophyll mass values in the given samples. When the dry and wet weights of the plant species are known, the content of chlorophyll a/b and the mass of crude extracts relative to the mass of the dry plant can be calculated and expressed as mg/gDW.

### Example 2: Preparation of Chlorophyllase-Treated Plant Leaf Extracts

Chlamydomonas reinhardtii chlorophyllase was produced as described previously (Molecules. 2015 Feb. 24; 20(3):3744-57; Biotechnol Appl Biochem. 2016 May; 63(3):371-7). Recombinant Chlamydomonas reinhardtii chlorophyllase was expressed, purified, and then lyophilized. The reaction mixture contained 0.5 mg of recombinant chlorophyllase, 650 μL of the reaction buffer (100 mM sodium phosphate, pH 7.4, and 0.24% Triton X-100), and 0.1 ml of crude extracts from leaves (100 mM chlorophyll). The reaction mixture was incubated at 37° C. for 30 min in a shaking water bath. The enzymatic reaction was stopped by adding 4 mL of ethanol, 6 mL of hexane, and 1 mL of 10 mM KOH, respectively. The reaction mixture was vortexed vigorously and centrifuged at 4000 rpm for 10 min to separate the two phases. The upper layer contained the untreated chlorophyll a/b; the bottom layer was chlorophyllase-treated crude extracts comprising chlorophyllide a/b. The chlorophyllase-treated crude extracts containing chlorophyllide a/b mixtures were then concentrated and the solvent was removed by evaporation under reduced pressure at 40° C. on a rotary evaporator. The concentrated crude extracts were processed by lyophilization, weighed, and stored at −80° C. for further experiments.

Chlorophyll was extracted from leaves of 9 plant species, including guava, sweet potato, banana, Chinese toona, logan, wax apple, mango, caimito, and cocoa. Ethanol crude extracts were treated with chlorophyllase to generate chlorophyllide, and then lyophilized in order to measure the weight. The results are listed in Table 1. Significantly, the most chlorophyll a level was observed in Chinese toona (9.8 mg/gDW), followed by mango (8.407 mg/gDW). The lowest chlorophyll a levels were present in banana (2.921 mg/gDW) and sweet potato (3.481 mg/gDW). For chlorophyll b, Chinese toona possessed the highest content (5.419 mg/gDW), followed by cocoa (4.485 mg/gDW) and mango (2.599 mg/gDW). The lowest levels of chlorophyll b were found in sweet potato (0.996 mg/gDW), banana (1.031 mg/gDW), and caimito (1.493 mg/gDW). Of the species analyzed, leaves of cocoa and caimito contained the highest level of ethanol crude extracts, at 412.65 and 397.62 mg/gDW, respectively. The lowest weight of ethanol crude extracts was obtained from sweet potato (43.175 mg/gDW), banana (47.76 mg/gDW), and wax apple (94.29 mg/gDW).

TABLE 1
The concentration of chlorophyll a/b extracted from leaves of plants

| | | | | Chlorophyllase- |
|---|---|---|---|---|
| | | | | treated |
| | | | | crude extracts |
| | | Chlorophyll | Chlorophyll | containing |
| | | a | b | chlorophyllide |
| Plant species | | (mg/gDW) | (mg/gDW) | a/b (mg/gDW) |
| Sweet | Ipomoea batatas | 3.481 | 0.996 | 43.17 |
| potato | | | | |
| Wax | Syzygium | 5.423 | 1.955 | 94.29 |
| apple | samarangense | | | |
| Guava | Psidium guajava | 5.219 | 1.493 | 124.39 |
| Banana | Musa paradisiaca | 2.921 | 1.031 | 47.76 |
| Chinese | Toona sinensis | 9.800 | 5.419 | 148.19 |
| toona | | | | |
| Logan | Dimocarpus | 7.044 | 1.903 | 183.15 |
| | longan | | | |
| Mango | Mangifera indica | 8.407 | 2.599 | 291.77 |
| Caimito | Pouteria Caimito | 5.218 | 1.493 | 397.62 |
| Cocoa | Theobroma cacao | 6.718 | 4.485 | 412.65 |

### Example 3: High-Performance Liquid Chromatography (HPLC) Analysis of Chlorophyll Catabolites

To analyze chlorophyll and chlorophyllide, the mixtures containing chlorophyllase-treated crude extracts were analyzed by using HPLC as described previously. Chlorophyllide was detected at a wavelength of 667 nm and identified by absorption spectra, peak ratios, and co-migration with authentic standards.

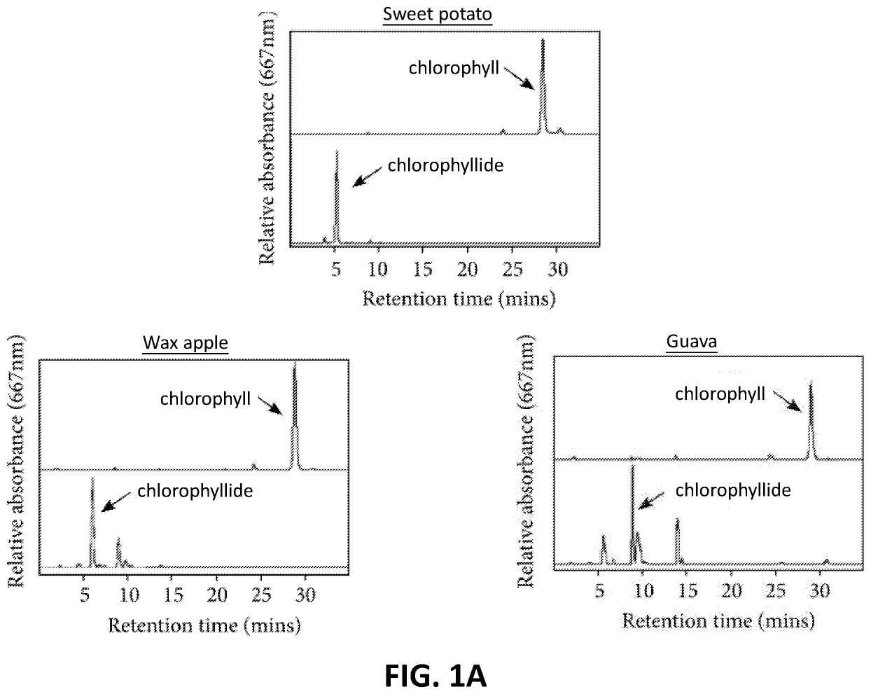

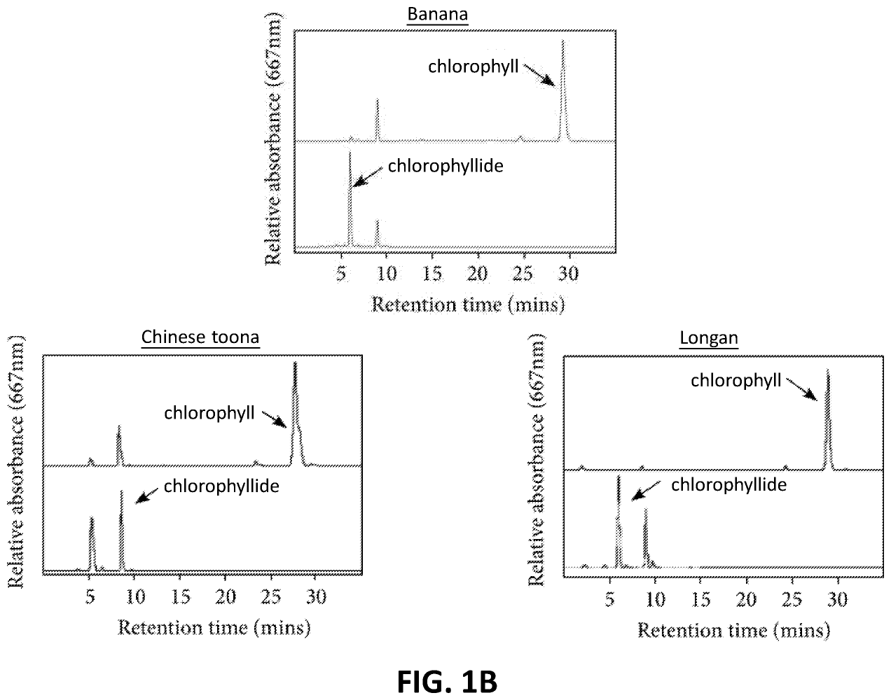

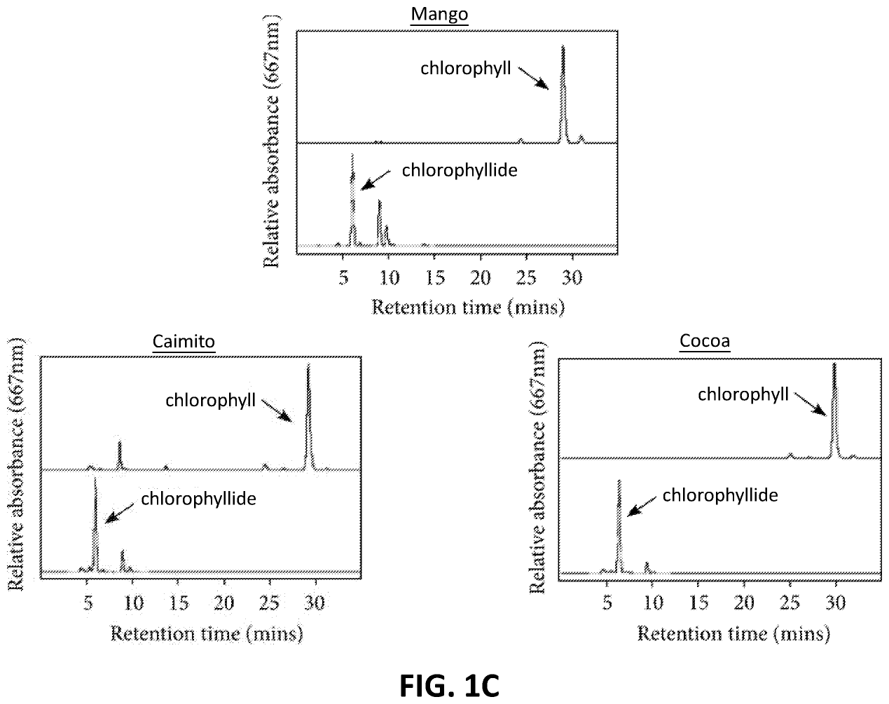

The HPLC separation system was applied to determine the amount of chlorophyll a/b and chlorophyllide a/b in crude extracts. Since the provision of commercial standards was limited, it was not possible to identify all peaks in all crude extracts by HPLC. Herein, the standards used in this study, including chlorophyll a, chlorophyll b, chlorophyllide a, and chlorophyllide b were selected based on our previous studies. HPLC results were obtained using mobile phases consisting of ethyl acetate/methanol/H₂O₂=44:50:6. Samples were quantified using photodiode array detection in the region 200-400 nm based on the retention times and UV spectra compared with the standards. FIGS. 1A to 1C show the HPLC profiles of guava, sweet potato, banana, Chinese toona, longan, wax apple, mango, caimito, and cocoa, respectively. The solvent system identified chlorophyll from 9 plant species within 30 min with a flow rate at 1 mL/min and detection at 667 nm. Chlorophyllide in crude extracts was detected within 10 min at 667 nm. According to the retention time, standards, and UV spectra, the peaks in FIGS. 1A to 1C were identified as chlorophyll and chlorophyllide.

### Example 4: Cell Cultures, Chemical Treatments, and Morphological Observations

Five eukaryotic cell lines were used to assess cytotoxicity in in vitro assays: human fibroblast cells (NIH/3T3), human breast cancer cell lines (MCF7 and MDA-MB-231), hepatocellular carcinoma cells (Hep G2), colorectal adenocarcinoma cells (Caco2), and glioblastoma cells (U-118 MG) were purchased from the American Type Culture Collection (ATCC) (Manassas, USA). Cells were cultured were in DMEM (Dulbecco's modified eagle medium) supplemented with 10% fetal bovine serum (FBS), Eagle's Minimum Essential Medium (EMEM), with 10% FBS and 0.01 mg/mL insulin, Leibovitz's L-15 Medium (L15) with 10% FBS, EMEM with 10% FBS, EMEM with 20% FBS, and DMEM with 10% FBS, respectively. The cells were maintained at 37° C. under a humidified atmosphere of 5% CO₂, except for MDA-MB-231. The cells were treated with increasing concentrations of chlorophyllide in ethanol extracts (50, 80, 100, 150, and 200 μg/mL), cultured in an incubator at 37° C. for 48 hr, and the cellular morphology was observed. Following incubation, the cells were observed under an inverted microscope.

### Example 5: Colorimetric MTT Viability Assay in Cancer Cell Lines

Cell viability was examined by the ability of the cells to cleave the tetrazolium salt MTT [3-(4,5-dimethylthiazol-2-yl)-2,5-diphenyl tetrazolium bromide] (Sigma Chem., St. Louis, Mo.) by the mitochondrial enzyme succinate dehydrogenase following a previously described procedure (Cold Spring Harbor protocols 2018, 2018(6): pdb prot095505). Cells were incubated at the temperature used to acclimatize cell lines. The background absorbance of the culture medium was subtracted from the measured absorbance. Cells (5×10⁴/well) were stimulated with different doses of chlorophyllide (50, 80, 100, 150, and 200 μg/mL). At the end of the incubation period, 24 hr post stimulation, 20 μL of the MTT solution was added per well. After treatment for 24 hr, supernatants were removed from the wells and 1% MTT solution was added to each well. The plates were incubated for 4 hr at 37° C. and the optical density was determined at 595 nm using a multi-well spectrophotometer (Multiskan, Thermo Fisher Scientific, Waltham, Mass.). All measurements made in the 96-well plates were performed using five technical replicates. In addition, cell viability was examined microscopically for the presence of cytopathic effect (CPE). The half-maximal inhibitory concentration (IC₅₀) was defined as the concentration required to inhibit cell viability by 50%. The IC₅₀ value and the standard error of the mean (SEM) were calculated using a non-linear regression curve contained in the SigmaPlot™ statistical software. A calculated selectivity index (SI) evaluated the relationship between cytotoxicity of cancer cells and normal cells. The SI was calculated from the IC₅₀ of normal NIH-3T3 versus cancer cells. The cytotoxicity effect was considered to have high selectivity for cancer cells if the SI exceeded 2. Values in Tables 2 and 3 were evaluated by linear regression analysis, and correlation coefficients between chlorophyll/chlorophyllide content and cytotoxic activity were calculated by Pearson's correlation coefficient. The values were between +1 (black color) and -1 (red color). The absolute value of correlation coefficient ranges 0.7-0.99, 0.4-0.69, 0.1-0.39 and 0.01-0.09, which was defined as highly, moderately, modestly and weakly correlations.

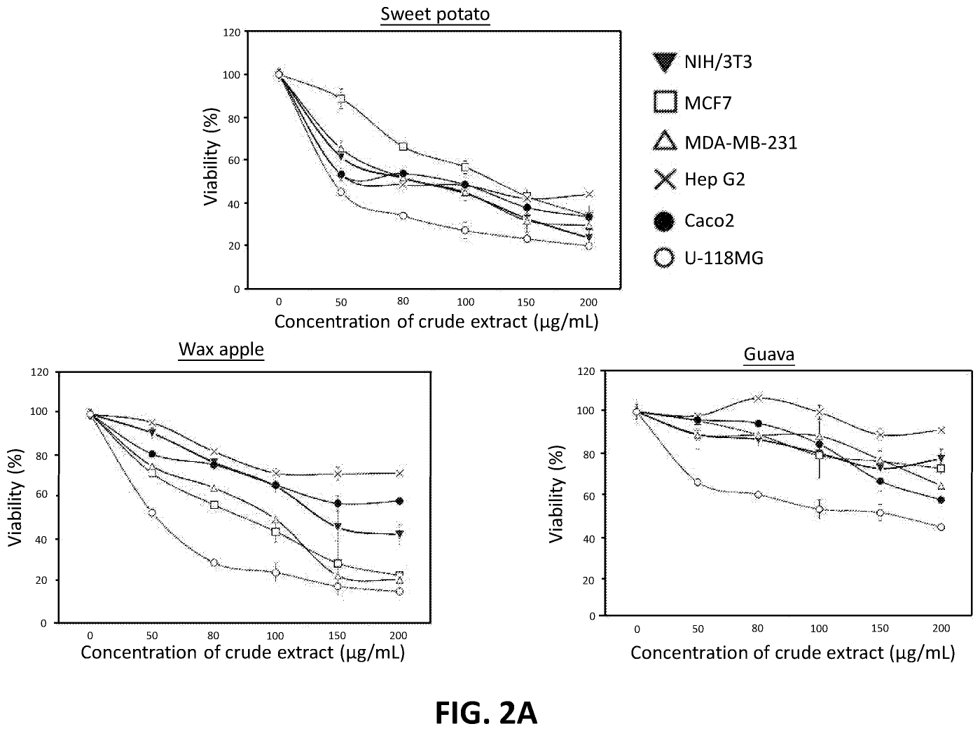

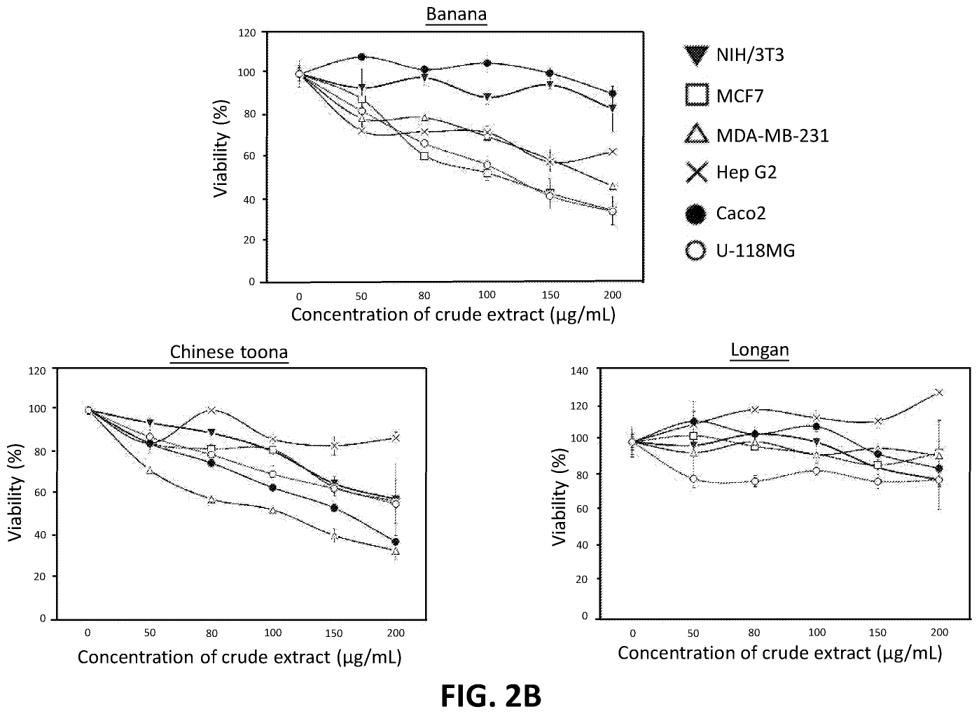

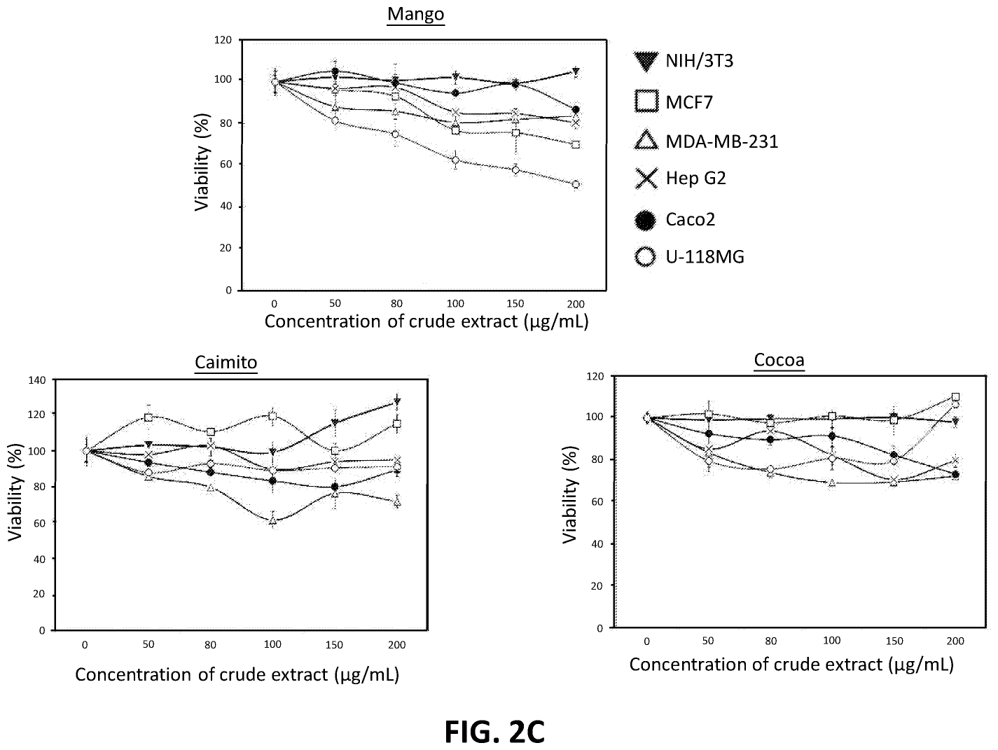

The cytotoxic effect of 9 chlorophyllase-treated ethanol crude extracts from plants against human fibroblast cells (NIH/3T3), human breast cancer cell lines (MC7 and MDA-MB-231), hepatocellular carcinoma cells (Hep G2), colorectal adenocarcinoma cells (Caco2), and glioblastoma cells (U-118 MG) were determined by MTT assay at a concentration range of 50-200 μg/mL. As shown in FIGS. 2A to 2C, chlorophyllase-treated ethanol crude extracts from guava induced the death of U-118 MG cells in a concentration-dependent manner with an IC₅₀ value of 134 μg/mL (P<0.01), while MCF-7, MDA-MB-231, and Caco2 cells displayed moderate viability in response to guava (IC_{50>200} μg/mL). For sweet potato, chlorophyllase-treated ethanol crude extracts induced a concentration-dependent cytotoxic response in all human cell lines tested, including NIH/3T3 cells. Compared with the other plants, sweet potato presented a lower IC₅₀ value, at 82.68, 122.29, 82.9, 63.73, 80.73, and 43.17 μg/mL in NIH/3T3, MCF-7, MDA-MB-231, Hep G2, Caco2, and U-118 MG cells, respectively. The cytotoxic effect of toona was similar to that of sweet potato, with a slightly higher IC₅₀ value, except for Hep G2 cells. Chlorophyllase-treated ethanol crude extracts from banana presented high levels of cytotoxicity against all tested cell lines, especially MDA-MB-231, Hep G2, and U-118 MG cells. With longan, the greatest cytotoxicity was found in Hep G2, Caco2, and U-118 MG cell lines, and no evident effects were found in MCF7 and MDA-MB-231 cells. For wax apple, significant and dose-dependent cytotoxicity was observed in MCF7, MDA-MB-231, and U-118 MG cells. In NIH/3T3 cells, mango, caimito, and cocoa presented no evidence of cytotoxicity. However, only small difference in ethanol crude extracts were observed between the effects of mango, caimito, and cocoa in MCF7, MDA-MB-231, Hep G2, and U-118 MG cells, with an IC_{50>200} μg/mL.

Based on the dose-response curve, the IC₅₀ of each extract was calculated, and these are summarized in Tables 2 and 3. MCF7 cells were more sensitive to chlorophyllide in ethanol extracts of wax apple and banana with an IC₅₀ of 88.87 and 104.41 μg/mL, respectively. MDA-MD-231 cells were most sensitive to sweet potato and wax apple, with IC₅₀ values of 82.9 and 97.83 μg/mL, respectively. In Hep G2 cell lines, sweet potato had the lowest IC₅₀ at 63.73 μg/mL, while those of other plants were nearly 200 μg/mL. In Caco2 cells, the IC₅₀ value of sweet potato was 80.73 μg/mL. U-118 MG cells, which represent the most sensitive of the tested cell lines, were responsive to sweet potato, wax apple, banana, and guava, with IC₅₀ values of 43.17, 52.64, 119.59, and 133.55 μg/mL, respectively (P<0.01).

Selectivity index (SI) is defined as the ratio between the IC₅₀ of each plant extract in cancerous and normal NIH/3T3 cells. An SI exceeding 2 was considered to indicate high selectivity. The SI values were calculated to verify the therapeutic potential of plant extracts. Banana had the highest SI value at 4.6, 4.02, 2.57, and 2.5 in MCF7, U-118 MG, MDA-MB-231, and Hep G2 cell lines, respectively. Wax apple and guava had the highest selectivity, with SI values of 2.75 and 2.37, respectively, in U-118 MG cell lines. Toona showed high selectivity towards MDA-MB-231 cell lines with an SI of 2.12. Among the extracts tested, sweet potato exhibited promising cytotoxicity with the lowest IC₅₀ values (43.17-82.9 μg/mL) in U-118 MG, Hep G2, Caco2, and MDA-MB-231 cells. However, the highest SI found for sweet potato was 1.915, in U-118 MG cell lines.

TABLE 2
IC₅₀ and SI values of chlorophyllase-treated crude extracts on cancer cell line

| | MCF7 | | MDA-MB-231 | | Hep G2 | | Caco2 | | U-118 MG | | NIH/3T3 |
|---|---|---|---|---|---|---|---|---|---|---|---|
| Plant species | IC₅₀ | SI | IC₅₀ | SI | IC₅₀ | SI | IC₅₀ | SI | IC₅₀ | SI | IC₅₀ |
| Sweet potato | 122.29 | 0.67 | 82.90 | 0.99 | 63.73 | 1.29 | 80.73 | 1.02 | 43.17 | 1.915 | 82.68 |
| Wax apple | 88.87 | 1.63 | 97.83 | 1.48 | >200 | 0.57 | >200 | 0.66 | 52.64 | 2.75 | 144.90 |
| Guava | >200 | 1.03 | >200 | 1.10 | >200 | 0.72 | >200 | 1.41 | 133.55 | 2.37 | >200 |
| Banana | 104.41 | 4.60 | 186.99 | 2.57 | 192.07 | 2.50 | N/A | 1.00 | 119.59 | 4.02 | >200 |
| Chinese toona | >200 | 1.02 | 107.24 | 2.12 | >200 | 0.74 | 154.63 | 1.47 | 206.01 | 1.10 | >200 |
| Logan | >200 | 0.72 | >200 | 0.56 | >200 | N/A | >200 | 0.99 | >200 | 1.28 | >200 |
| Mango | >200 | N/A | >200 | N/A | >200 | N/A | >200 | N/A | >200 | N/A | >200 |
| Caimito | >200 | N/A | >200 | N/A | >200 | N/A | >200 | N/A | >200 | N/A | >200 |
| Cocoa | >200 | N/A | >200 | N/A | >200 | N/A | >200 | N/A | >200 | N/A | >200 |

TABLE 3
IC₅₀ and SI values of chlorophyllide from chlorophyllase-treated crude extracts on cancer cell line

| | MCF7 | | MDA-MB-231 | | Hep G2 | | Caco2 | | U-118 MG | | NIH/3T3 |
|---|---|---|---|---|---|---|---|---|---|---|---|
| Plant species | IC₅₀ | SI | IC₅₀ | SI | IC₅₀ | SI | IC₅₀ | SI | IC₅₀ | SI | IC₅₀ |
| Sweet potato | 11.02 | 0.75 | 8.60 | 0.96 | 9.00 | 0.92 | 8.75 | 0.94 | 6.45 | 1.28 | 8.26 |
| Wax apple | 6.98 | 1.38 | 7.13 | 1.36 | 15.43 | 0.63 | 11.95 | 0.81 | 5.28 | 1.83 | 9.66 |
| Guava | 12.87 | 1.03 | 12.01 | 1.10 | 18.44 | 0.72 | 9.39 | 1.41 | 5.60 | 2.37 | 13.26 |
| Banana | 10.82 | 3.66 | 14.90 | 2.66 | 15.86 | 2.50 | >20 | 1.00 | 11.10 | 3.57 | >20 |
| Chinese toona | 14.67 | 1.02 | 8.14 | 1.84 | 20.32 | 0.74 | 10.36 | 1.45 | 13.62 | 1.10 | 15.02 |
| Logan | 19.19 | 0.72 | >20 | 0.56 | >20 | N/A | 13.51 | 1.03 | 10.88 | 1.28 | 13.87 |
| Mango | >20 | N/A | >20 | N/A | >20 | N/A | >20 | N/A | 15.51 | N/A | >20 |
| Caimito | >20 | N/A | 15.13 | N/A | >20 | N/A | 16.74 | N/A | >20 | N/A | >20 |
| Cocoa | >20 | N/A | >20 | N/A | >20 | N/A | >20 | N/A | >20 | N/A | >20 |

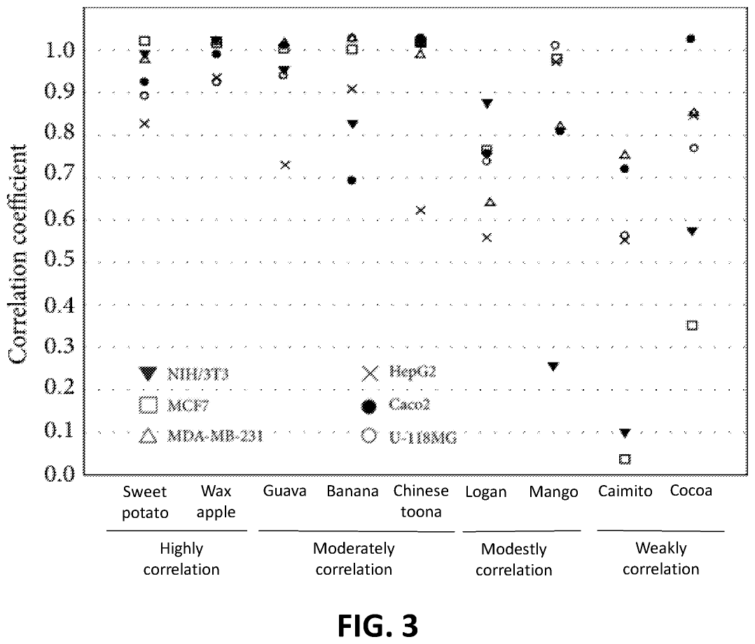

Values in Tables 2 and 3 were evaluated by linear regression analysis, and correlation coefficient was calculated by Pearson's correlation coefficient and shown in Table 4 and FIG. 3. It was found that the correlation between chlorophyll/chlorophyllide content and cytotoxic activity differed from plant to plant. According to the correlation coefficients, 9 plants were divided into 4 groups. First, the highly correlations (correlation coefficient: 0.7-0.99) were found in sweet potato and wax apple. Guava, banana and toona were classified into group 2 which the correlation was located between moderately (correlation coefficient: 0.4-0.69) to highly correlation. Longan and mango were belonged to group 3. In this group, the correlation was modestly (correlation coefficient: 0.1-0.39) to moderately correlation. The weakly correlations (correlation coefficient: 0.01-0.09) were observed at caimito and cacao (group 4). Therefore, ethanol crude extracts of each plant have other unique and functional components which may affect the cytotoxic function of chlorophyllide.

TABLE 4
The values of correlation coefficient between MTT activity and chlorophyllide contents

| | | Sweet | | Chinese | | Wax | | | |
|---|---|---|---|---|---|---|---|---|---|
| | Guava | potato | Banana | toona | Logan | apple | Mango | Caimito | Cocoa |
| NIH/3T3 | −0.902 | −0.945 | −0.774 | −0.983 | −0.831 | −0.976 | −0.175 | −0.057 | −0.519 |
| MCF7 | −0.954 | −0.975 | −0.955 | −0.983 | −0.712 | −0.967 | −0.932 | 0.014 | −0.302 |
| MDA-MB-231 | −0.971 | −0.933 | −0.986 | −0.959 | −0.584 | −0.976 | −0.768 | −0.709 | −0.797 |
| Hep G2 | −0.666 | −0.770 | −0.859 | −0.572 | −0.497 | −0.878 | −0.922 | −0.497 | −0.789 |
| Caco2 | −0.961 | −0.876 | −0.634 | −0.995 | −0.701 | −0.937 | −0.758 | −0.676 | −0.975 |
| U-118 MG | −0.886 | −0.839 | −0.982 | −0.982 | −0.685 | −0.870 | −0.964 | −0.507 | −0.713 |

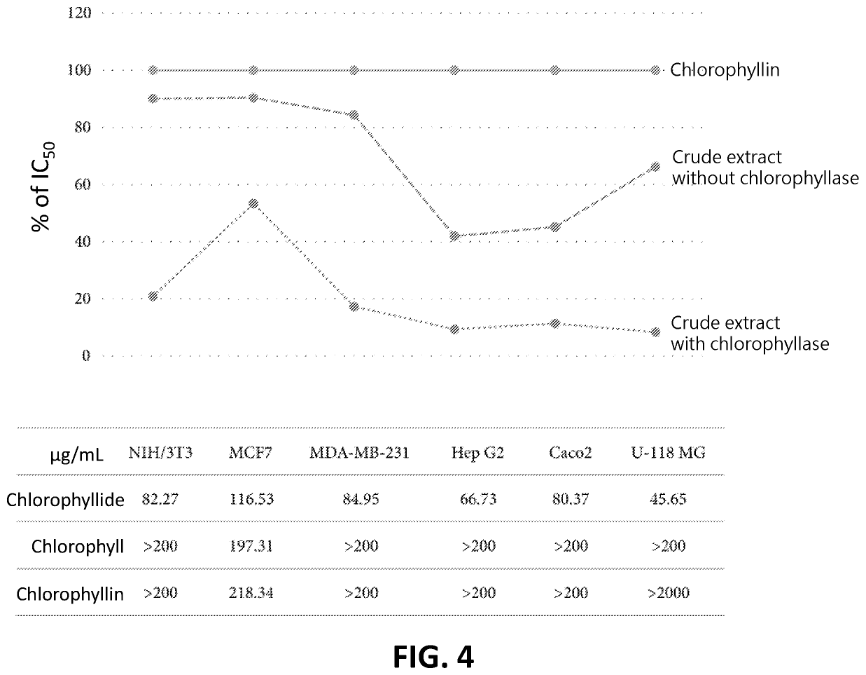

To confirm that chlorophyllide in ethanol extracts has an important effect on cell viability, the cytotoxicity of chlorophyll and chlorophyllide in sweet potato leaf ethanol extracts and of chlorophyllin against MCF7, MDA-MD-231, Hep G2, Caco2, and U-118 MG cell lines were compared. Chlorophyll, chlorophyllide, and chlorophyllin were analyzed in an MTT assay at concentrations between 0 and 200 μg/mL. As shown in FIG. 4, the results indicated that chlorophyllase-treated crude extract from sweet potato exhibited promising cytotoxicity against MCF7, MDA-MD-231, Hep G2, Caco2, and U-118 MG cell lines, with IC₅₀ values of 116.53, 84.95, 66.73, 80.37, and 45.65 μg/mL, respectively. Chlorophyll possessed only moderate cytotoxicity against MCF7 cells, with an IC₅₀ of 197.31 μg/mL. Chlorophyllin demonstrated low activity towards MCF7 cells, with an IC₅₀ of 218.34 μg/mL. These results were generally consistent with those observed in the screening test, confirming that U-118 MG, Hep G2, Caco2, and MDA-MB-231 cells were sensitive to chlorophyllase-treated ethanol crude extracts from sweet potato, for which the lowest IC₅₀ values were found. Chlorophyll and chlorophyllin presented poor activity and selectivity compared with chlorophyllide.

### Example 6: Free Radical Scavenging Assay

The DPPH assay was used to evaluate the free radical-scavenging of chlorophyllide. Briefly, DPPH (8 mg) was dissolved in methanol (100 mL) to obtain a stock solution of 80 μg/mL. Then, 2.95 mL of the working solution was mixed with 50 μL of sample. After incubation in a dark at room temperature for 20 min, the absorbance was measured at 517 nm. The DPPH scavenging effect (%) was determined using the following formula:

$$Kd ( \% ) = \frac{Ac - ( Ai - Aj )}{Ac} \times 1 \; 00 \% ;$$

where Ac was the absorbance of the blank control, Ai was the absorbance in the presence of the samples, and Aj was the absorbance of the samples alone. Vitamin B2 was used as a reference standard compound. The EC₅₀ value, which is the concentration that can inhibit 50% of DPPH free radicals, was obtained by extrapolation from regression analysis.

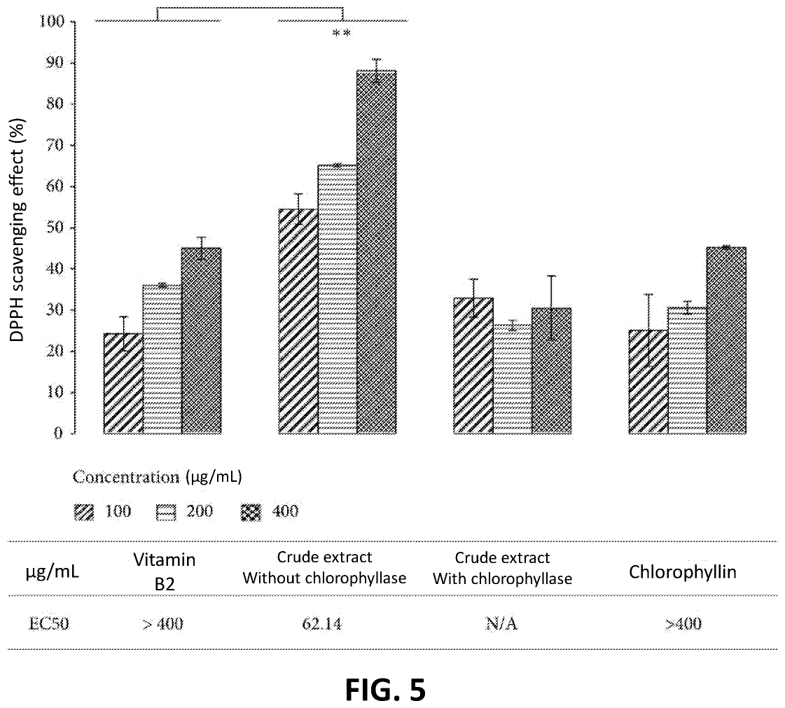

The anti-oxidant capacities of chlorophyll and chlorophyllide from sweet potato leaf ethanol extracts and chlorophyllin were compared by DPPH assay. FIG. 5 shows the DPPH radical scavenging activity of chlorophyll, chlorophyllin, and the positive control vitamin B2 increased in a dose-dependent manner. The scavenging rates of chlorophyll reached 52.95, 65.11, and 88.62% at 100, 200, and 400 μg/mL, respectively, which were higher than those observed for vitamin B2. The scavenging rates of chlorophyllin were 25.68, 30.58, and 45.34%, respectively. The scavenging rate of chlorophyllide reached 31.01% at 100 μg/mL. When the concentration increased to 200 μg/mL, the scavenging activity of chlorophyllide (26.92%) was similar to that observed with 100 μg/mL of vitamin B2 (28.05%); this remained stable (26.09%) with 400 μg/mL of chlorophyllide. The EC₅₀ was calculated by SigmaPlot software and the result indicated that the EC₅₀ values of vitamin B2 and chlorophyllin exceeded 400 μg/mL, while that of chlorophyll was 62.14 μg/mL.

### Example 7: Doxorubicin Resistance Assay

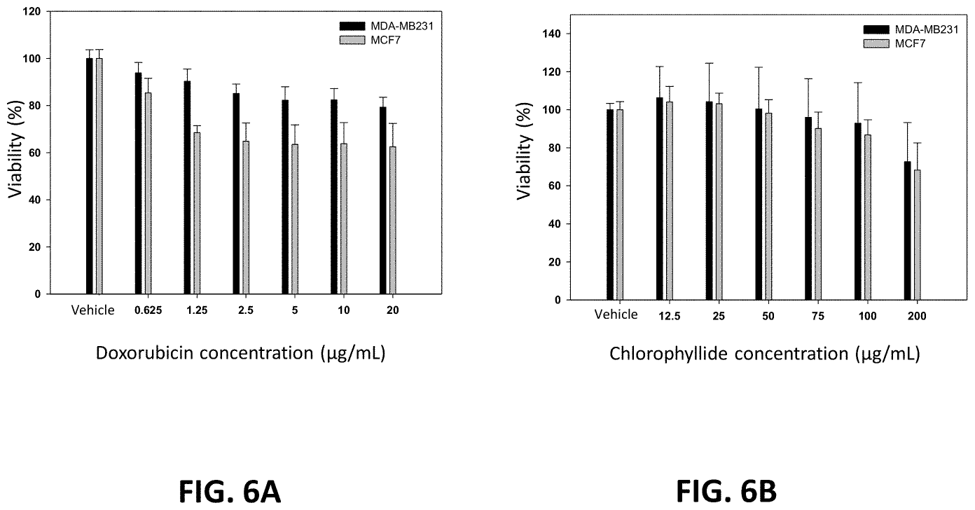

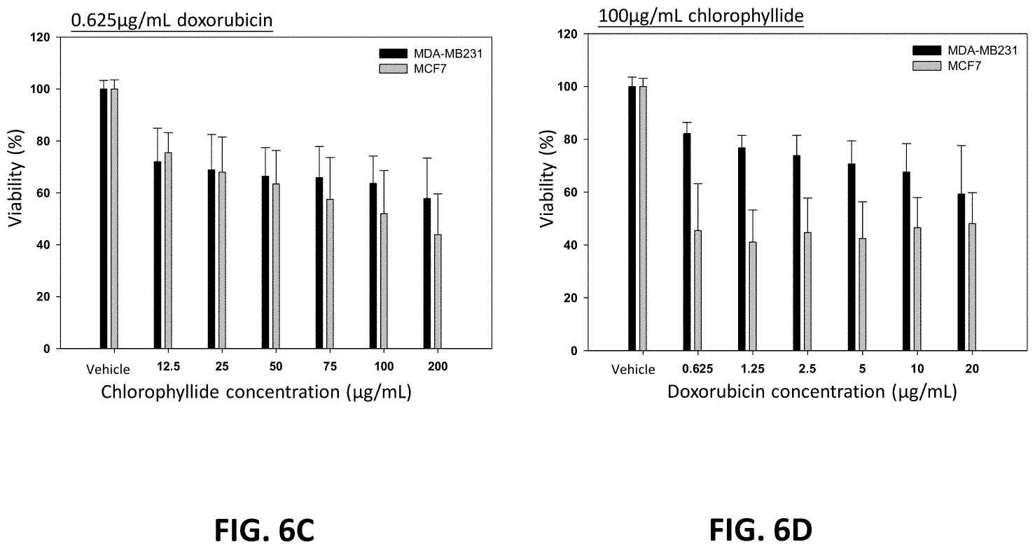

The colorimetric MTT viability assay was performed as above, except for the chemicals for stimulation. As shown in FIG. 6A, merely in the presence of 0.625 μg/mL doxorubicin, there were no cytotoxic effects on MCF7 breast cancer cells and MDA-MB-231 breast cancer cells. As shown in FIG. 6B, merely in the presence of the chlorophyllase-treated hexane crude extract from sweet potato containing 100 μg/mL chlorophyllide, there were no cytotoxic effects on MCF7 breast cancer cells and MDA-MB-231 breast cancer cells.

The colorimetric MTT viability assay was performed with the treatment of 0.625 μg/mL doxorubicin in combination of the treatment of the chlorophyllase-treated hexane crude extract from sweet potato containing various concentrations of chlorophyllide. As shown in FIG. 6C, while doxorubicin was fixed at 0.625 μg/mL, there was a decrease of cellular survival rates with an increase of the chlorophyllide concentrations in the chlorophyllase-treated hexane crude extract from sweet potato from 12.5 to 200 μg/mL. This indicated the combination of doxorubicin and a chlorophyllase-treated hexane crude extract from sweet potato containing chlorophyllide creates a synergistic effect on the activity of cancer cell cytotoxicity.

The colorimetric MTT viability assay was performed with the treatment of the chlorophyllase-treated hexane crude extract from sweet potato containing 100 μg/mL chlorophyllide in combination of the treatment of various concentrations of doxorubicin. As shown in FIG. 6D, while chlorophyllide was fixed at 100 μg/mL, there was no change of cellular survival rates with an increase of the doxorubicin concentrations from 0.625 to 20 μg/mL. This indicated the foregoing synergistic effect on the activity of cancer cell cytotoxicity was resulted from chlorophyllide, not doxorubicin.

### Example 8: Differential Gene Expression Analysis

The gene expression profiles of the MCF7 breast cancer cells treated with chlorophyllide or MDA-MB-231 breast cancer cells treated with chlorophyllide and the corresponding untreated cells were analyzed by using next generation sequencing (NGS).

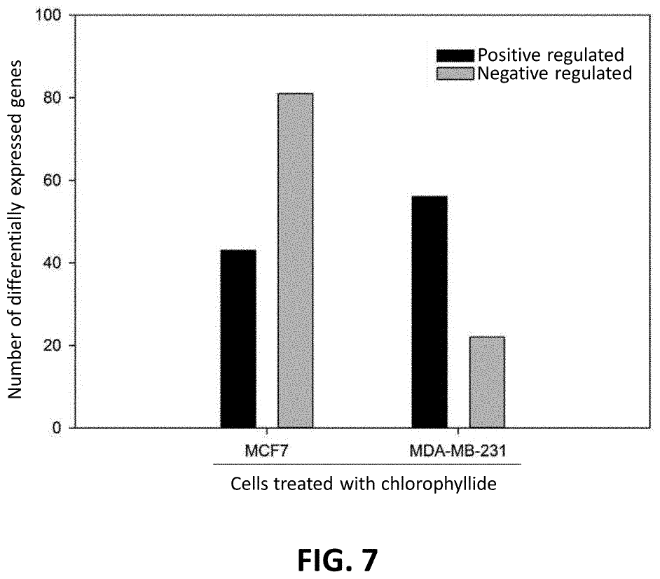

FIG. 7 shows 124 differentially expressed genes including 43 positive regulated genes and 81 negative regulated genes were found in MCF7 breast cancer cells treated with chlorophyllide relative to reference untreated MCF7 breast cancer cells; 77 differentially expressed genes including 56 positive regulated genes and 21 negative regulated genes were found in MDA-MB-231 breast cancer cells treated with chlorophyllide relative to reference untreated MDA-MB-231 breast cancer cells.

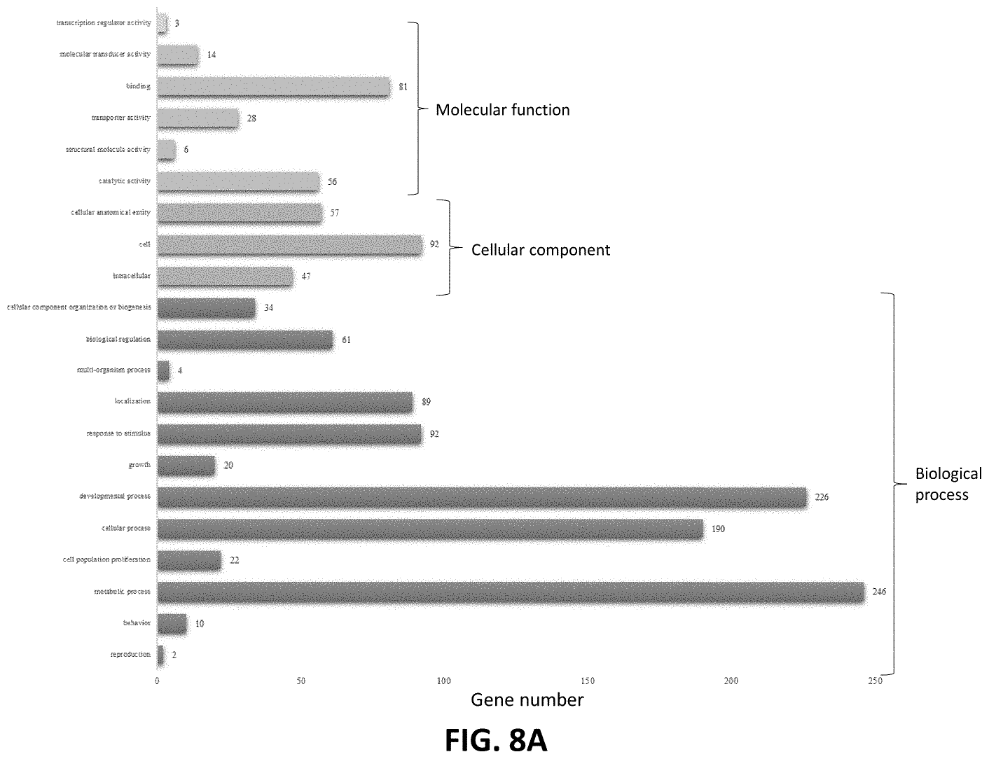

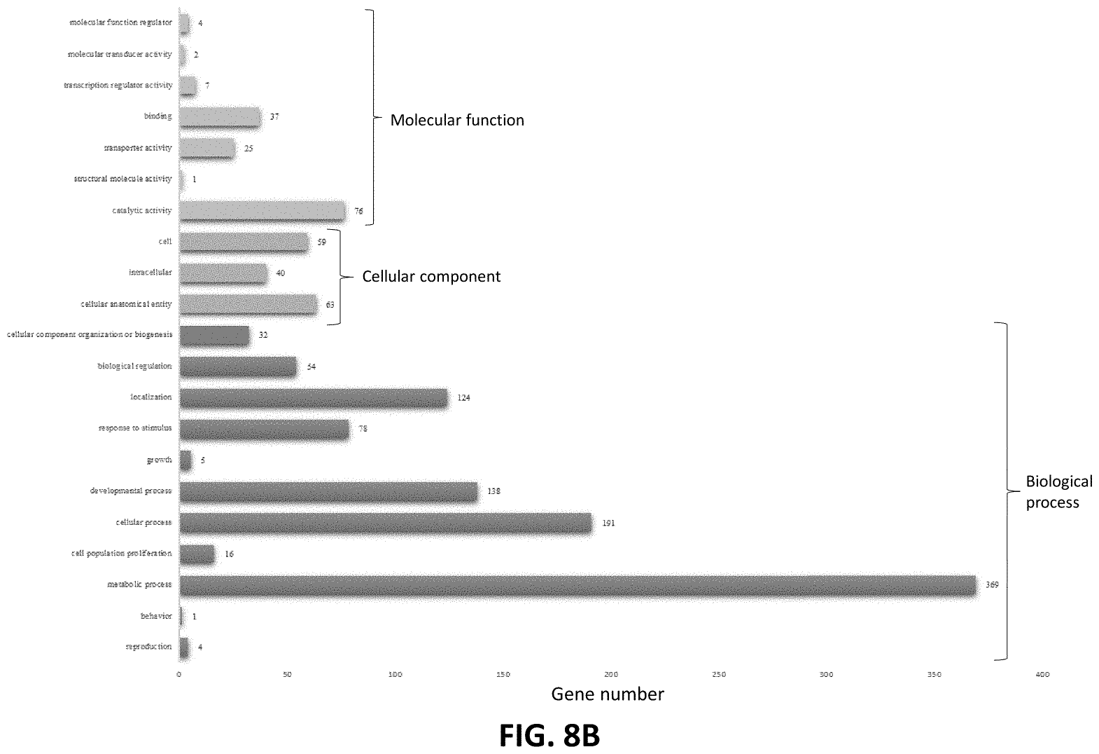

The Gene Ontology analysis was performed to classify the foregoing 2 differentially expressed gene groups. FIG. 8A shows that the differentially expressed gene group identified from MCF7 breast cancer cells treated with chlorophyllide relative to reference untreated MCF7 breast cancer cells were primarily classified into the subdomain “binding” of the main domain “molecular function”, the subdomain “cell” of the main domain “cellular component”, and the subdomain “metabolic process” of the main domain “biological process”. FIG. 8B shows that the differentially expressed gene group identified from MDA-MB-231 breast cancer cells treated with chlorophyllide relative to reference untreated MDA-MB-231 breast cancer cells were primarily classified into the subdomain “catalytic activity” of the main domain “molecular function”, the subdomain “cellular ana” of the main domain “cellular component”, and the subdomain “metabolic process” of the main domain “biological process”. As above, the subdomain “metabolic process” was the most significant subdomain of the foregoing 2 differentially expressed gene groups.

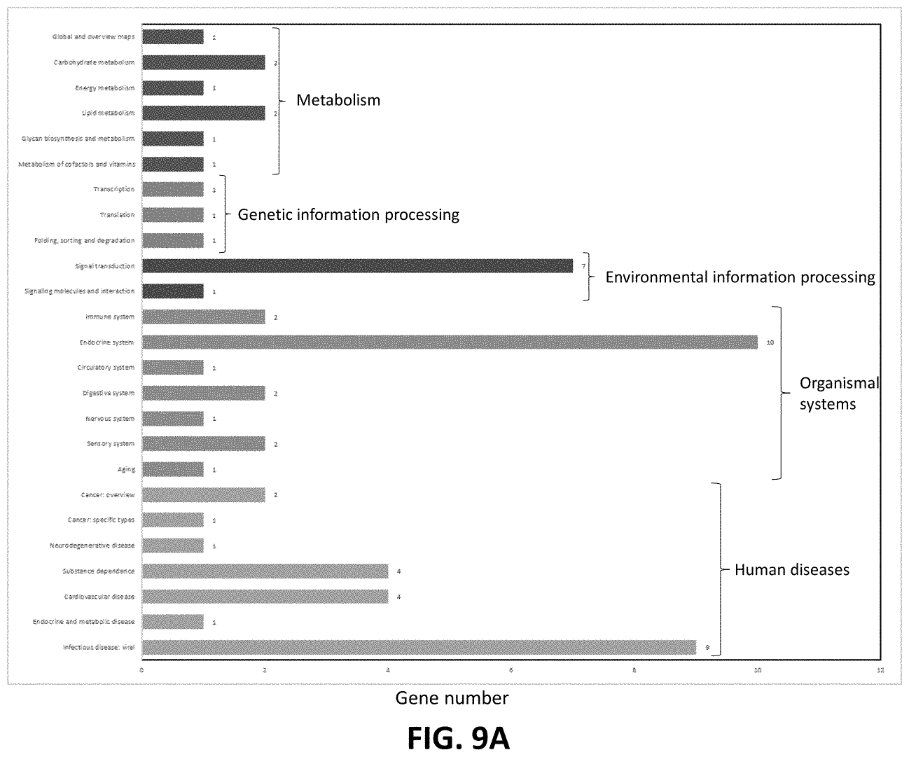

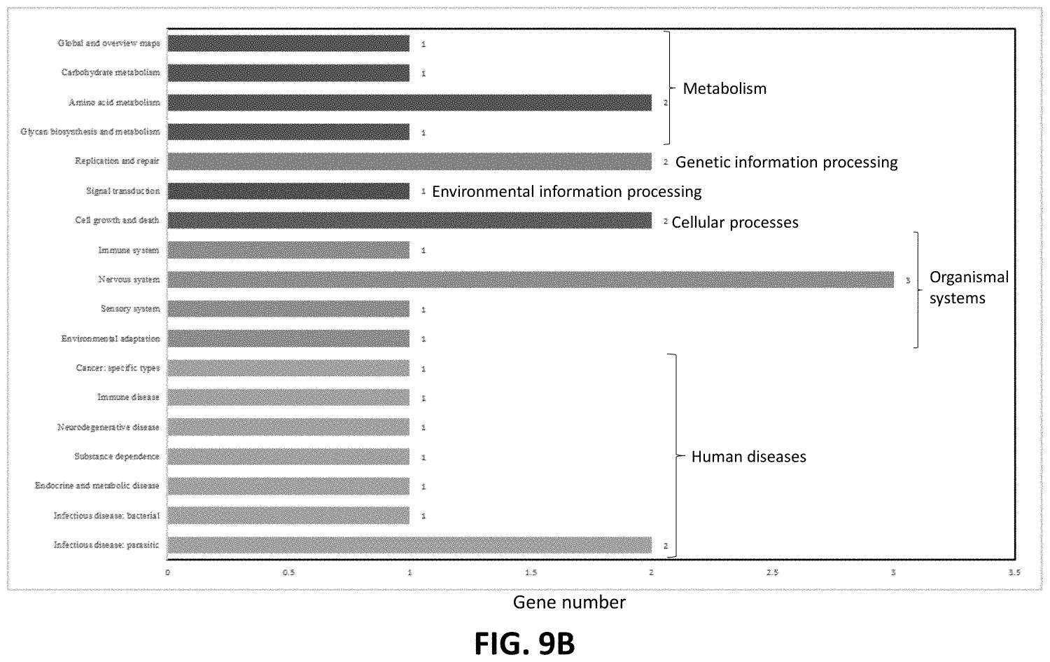

The KEGG pathway enrichment analysis was also performed to classify the foregoing 2 differentially expressed gene groups. In the KEGG pathway enrichment analysis, all of the foregoing differentially expressed genes were classified according to different KEGG pathways including “metabolism”, “genetic information processing”, “environmental information processing”, “cellular processes”, “organismal systems”, and “human diseases”. FIG. 9A shows the differentially expressed gene group identified from MCF7 breast cancer cells treated with chlorophyllide relative to reference untreated MCF7 breast cancer cells were primarily classified into “human diseases” and secondly classified into “organismal systems”, but not classified into “cellular processes”. Among the domain “human diseases”, 17 differentially expressed genes were related to the subdomain “infectious disease viral”, 4 differentially expressed genes were related to the subdomain “substance dependence”, and 4 differentially expressed genes were related to the subdomain “cardiovascular disease”. Among the domain “organismal systems”, 10 differentially expressed genes were related to the subdomain “endocrine system”, 2 differentially expressed genes were related to the subdomain “immune system”, 2 differentially expressed genes were related to the subdomain “digestive system”, and 2 differentially expressed genes were related to the subdomain “sensory system”. FIG. 9B shows the differentially expressed gene group identified from MDA-MB-231 breast cancer cells treated with chlorophyllide relative to reference untreated MDA-MB-231 breast cancer cells were primarily classified into “human diseases”. Among the domain “human diseases”, 2 differentially expressed genes were related to the subdomain “infectious disease parasitic”. Additionally, the differentially expressed gene group identified from MDA-MB-231 breast cancer cells treated with chlorophyllide relative to reference untreated MDA-MB-231 breast cancer cells were also related to the subdomain “nervous system” of the domain “organismal systems”.

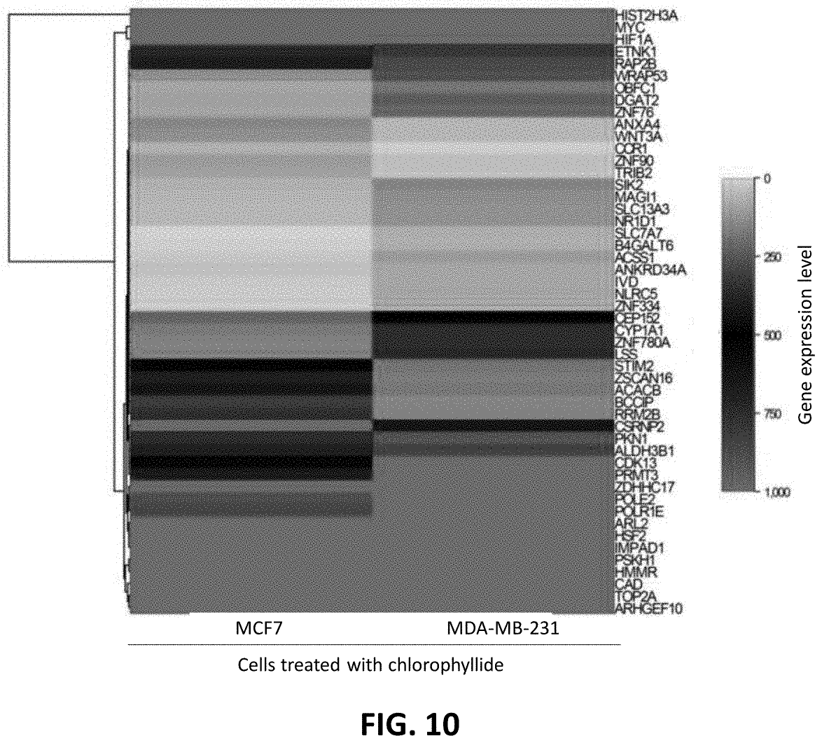

As shown in FIG. 10, 50 candidate genes were chosen from the differentially expressed genes identified from MCF7 breast cancer cells treated with chlorophyllide relative to reference untreated MCF7 breast cancer cells and the differentially expressed genes identified from MDA-MB-231 breast cancer cells treated with chlorophyllide relative to reference untreated MDA-MB-231 breast cancer cells. Then, among the 50 candidate genes, CCR1, STIM2, ETNK1, RAP2B, and TOP2A were used as target genes in real-time polymerase chain reaction (RT-PCR). The RT-PCR result is shown in Table 5.

TABLE 5
The gene expression level of the target genes
detected by using RT-PCR

| | Log₂ | MCF7 breast | MDA-MB-231 breast |
|---|---|---|---|
| Gene | fold | cancer cells treated | cancer cells treated |
| name | change | with chlorophyllide | with chlorophyllide |
| CCR1 | 5.954 | 6.798 | 10.079 |
| STIM2 | 2.783 | 6.125 | 3.5088 |
| ETNK1 | 2.181 | 1.98727 | 5.954 |
| RAP2B | 2.375 | 0.58816 | 3.694 |
| MAGI1 | (2.307) | 3.12595 | 1.3977 |
| NLRC5 | (5.824) | 0.68403 | 5.527 |
| SLC7A7 | (22.208) | 0.31069 | 1.159 |
| PKN1 | (2.520) | 1.16696 | 0.5819 |
| TOP2A | (2.230) | 1.57446 | 11.01549 |

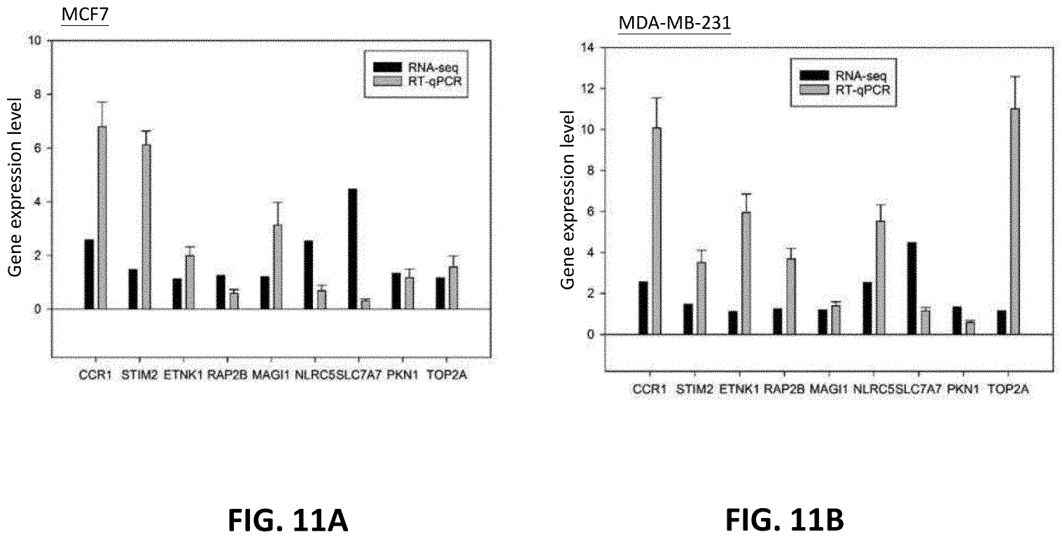

As shown in FIG. 11A, in MCF7 breast cancer cells treated with chlorophyllide, the gene expression levels of CCR1, STIM1, and MAGI1 detected by using RT-PCR were higher than those detected by using RNA-seq, but the gene expression levels of NLRC5 and SLC7A7 detected by using RT-PCR were lower than those detected by using RNA-seq. As shown in FIG. 11B, in MDA-MB-231 breast cancer cells treated with chlorophyllide, the gene expression levels of CCR1, ETNK1, RAP2B, NLRC5, and TOP2A detected by using RT-PCR were higher than those detected by using RNA-seq, but the gene expression level of SLC7A7 detected by using RT-PCR was lower than those detected by using RNA-seq.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for treating cancer, comprising:
administering a therapeutically effective dose of chlorophyllide to a subject in need thereof.

2. The method as claimed in claim 1, wherein the chlorophyllide administering step comprises:
administering a product obtained by treating a plant leaf extract with chlorophyllase to the subject in need thereof, wherein the product comprises the therapeutically effective dose of chlorophyllide.

3. The method as claimed in claim 2, wherein the product is produced by the steps of:
providing plant leaves;
performing extraction on the plant leaves with a solvent to obtain a crude extract; and
treating the crude extract with chlorophyllase to obtain the product.

4. The method as claimed in claim 3, wherein the solvent is ethanol or hexane.

5. The method as claimed in claim 1, wherein the cancer is breast cancer, liver cancer, colon adenocarcinoma, glioblastoma, lung cancer, buccal cancer, stomach cancer, colorectal cancer, nasopharyngeal cancer, skin cancer, kidney cancer, brain cancer, prostate cancer, ovarian cancer, cervical cancer, intestinal cancer, or bladder cancer.

6. The method as claimed in claim 5, wherein the cancer is drug-resistant cancer.

7. The method as claimed in claim 6, wherein the cancer is anthracycline-resistant cancer.

8. The method as claimed in claim 7, wherein the anthracycline-resistant cancer is doxorubicin-resistant cancer, daunorubicin-resistant cancer, arubicin-resistant cancer, epirubicin-resistant cancer, idarubicin-resistant cancer, valrubicin-resistant cancer, or mitoxantrone-resistant cancer.

9. The method as claimed in claim 5, wherein the cancer is triple-negative breast cancer.

10. A pharmaceutical composition, comprising:
a therapeutically effective dose of chlorophyllide; and
a therapeutically effective dose of anthracycline.

11. The pharmaceutical composition as claimed in claim 10, further comprising:
a product obtained by treating a plant leaf extract with chlorophyllase, wherein the product comprises the therapeutically effective dose of chlorophyllide.

12. The pharmaceutical composition as claimed in claim 10, wherein the anthracycline is doxorubicin, daunorubicin, arubicin, epirubicin, idarubicin, valrubicin, or mitoxantrone.

13. A method for treating cancer, comprising:
administering the pharmaceutical composition as claimed in claim 10 to a subject in need thereof.

14. The method as claimed in claim 13, wherein the therapeutically effective dose of chlorophyllide is from 12.5 to 100 μg/mL, and the therapeutically effective dose of anthracycline is from 0.625 to 20 μg/mL.

15. The method as claimed in claim 14, wherein the pharmaceutical composition further comprises:
a product obtained by treating a plant leaf extract with chlorophyllase, wherein the product comprises the therapeutically effective dose of chlorophyllide.

16. The method as claimed in claim 15, wherein the product is produced by the steps of:
providing plant leaves;
performing extraction on the plant leaves with a solvent to obtain a crude extract; and
treating the crude extract with chlorophyllase to obtain the product.

17. The method as claimed in claim 13, wherein the anthracycline is doxorubicin, daunorubicin, arubicin, epirubicin, idarubicin, valrubicin, or mitoxantrone.

18. The method as claimed in claim 16, wherein the cancer is breast cancer, liver cancer, colon adenocarcinoma, glioblastoma, lung cancer, buccal cancer, stomach cancer, colorectal cancer, nasopharyngeal cancer, skin cancer, kidney cancer, brain cancer, prostate cancer, ovarian cancer, cervical cancer, intestinal cancer, or bladder cancer.

19. The method as claimed in claim 16, wherein the cancer is anthracycline-resistant cancer.

20. The method as claimed in claim 16, wherein the plant is sweet potato, the solvent is hexane, the anthracycline is doxorubicin, and the cancer is breast cancer.
